(19) United States
(12) Patent Application Publication
(10) Pub. No.: US 2021/0337789 A1
(43) Pub. Date: Nov. 4, 2021

(54) Derivatives of m-Guaiacol, Their Preparation and Their Uses

(71) Applicants: Universite de Caen Normandie, Caen (FR); LABEO, Saint Contest (FR)

(72) Inventors: Sylvain Rault, Moult (FR); Christophe Rochais, Caen (FR); Stéphane Lemaitre, Manvieux (FR); Feng Xiao, Caen (FR); Peggy Suzanne, Mondeville (FR); Patrick Dallemagne, Seulline (FR); Sophie Castagnet, Caen (FR); Albertine Leon-Seck, Hérouville-Saint-Clair (FR)

(73) Assignees: Universite de Caen Normandie, Caen (FR); LABEO, Saint Contest (FR)

(21) Appl. No.: 17/257,396

(22) PCT Filed: Jul. 2, 2019
(86) PCT No.: PCT/EP2019/067774

§ 371 Date: Dec. 31, 2020

(30) Foreign Application Priority Data
Jul. 3, 2018 (FR) 1856125

(51) Int. Cl. A01N 31/16; A61K 31/085

(52) U.S. Cl. CPC A01N 31/16; A61K 31/085

(57) ABSTRACT

The invention concerns derivatives of m-guaiacol, their preparation and their uses as biocides, in particular as antibacterials or disinfectants.

The invention concerns derivatives of m-guaiacol, their preparation and their uses as biocides, in particular as antibacterials or disinfectants.

Most cosmetic and dermopharmaceutical hygiene products contain one or several microbicide substances in their formula. Indeed, the raw materials used for manufacturing these products are only rarely perfectly sterile and the end products are too fragile (perfume, biological active ingredients, vitamins) to support sterilisation after packaging. Furthermore, and contrary to food products or to pharmaceutical products, cosmetic products are not obligated to carry an expiry date: therefore they can and must be stable and in perfect condition—including microbiologically—for a very long time.

Chemical preservatives used in these products fulfil this protective role. The choice of antimicrobial substances or biocide molecules that could be used is strictly governed by different legislations of European, American and Asian countries.

Triclosan, chlorinated bicycle, is currently one of the most used antibacterial preservatives in the cosmetic and food industry.

This compound is currently used as biocide and is in particular present in the composition of numerous hygiene and cosmetic products (soaps, shower gels, toothpastes, deodorants, mouthwashes, moisturisers, etc.). In this type of preparation, this molecule is used as an additive or active ingredient, as is the case particularly in a very large majority of soaps and antibacterial solutions. For these same properties, this molecule is also used to impregnate or be included in different plastic raw materials (food packaging, bin bags, sponges, etc.). It is also used as disinfectant and is widely used in care units and farming. This generalised use of triclosan for more than 40 years is currently being questioned by a certain number of scientific studies, in particular for fear of the appearance or the proliferation of bacteria resistant to both biocides and antibiotics. This molecule also has been described as potentially carcinogenic and is currently suspected of being an endocrine disruptor. Over the last few years, its removal from a large number of products currently consumed, in particular cosmetic products, has been requested in particular by the Food and Drug Administration (FDA). The bicyclic chemical structure of this molecule could be at the origin of these interaction phenomena with the hormone system.

A family of derivatives of m-guaiacol has now been developed showing a strong biocide activity on different bacterial strains (Gram+ et Gram−), and in particular an antibacterial action in the same range of concentration as the reference molecule, triclosan, while being less cytotoxic than the latter, even non-cytotoxic.

Of a different nature (in particular, due to their monocyclic, and therefore non-bicyclic structure), the molecules developed in the scope of this programme appear to be an alternative to triclosan.

Furthermore, the compounds of the invention are of simple chemical structure, which is easy to synthesise, at a reasonable cost (in particular, in an industrial environment).

Thus, according to a first aspect, the invention concerns the use of a compound of following formula (I) as biocide:

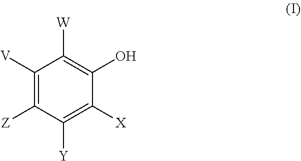

wherein:
V represents:
a group —OR wherein R is a linear or branched alkyl in C₁ to C₄, optionally substituted by at least one halogen, R being in particular a methyl or a perhalogenoalkyl in C₁ to C₄; or
a halogen;

W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;

at least one of W, X, Y and Z representing a halogen,

as well as its pharmaceutically acceptable salts.

According to a particular embodiment, V represents a group —OR as defined previously, or I.

According to a particular embodiment, the compound of formula (I) is not the following formula:

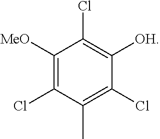

According to a particular embodiment, the compound of formula (I) is not one of the following formulae:

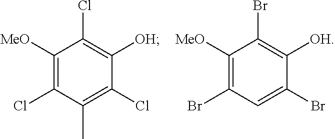

According to a particular embodiment, V represents a halogen, in particular I, and in particular:
W and/or Y represent H; and/or
X and/or Z represent a halogen, in particular F.

According to a particular embodiment, V represents I.

According to a particular embodiment, the invention concerns the use of a compound of following formula (I) as biocide:

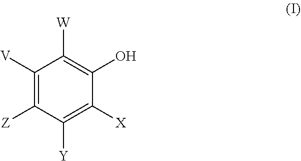

wherein:
V represents:
a group —OR, wherein R is a linear or branched alkyl in C₁ to C₄, optionally substituted by at least one halogen, R being in particular a methyl or a perhalogenoalkyl in C₁ to C₄; or
I; V representing preferably a group —OR;

W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;

at least one of W, X, Y and Z representing a halogen,

the following compound being excluded:

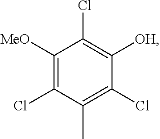

as well as its pharmaceutically acceptable salts.

According to a particular embodiment, W does not represent a halogen, in particular Cl, and Y does not represent a linear or branched alkyl in C₁ to C₄, in particular Me.

According to a particular embodiment, when Y represents a linear or branched alkyl in C₁ to C₄, in particular Me, then W, X and Z do not represent a halogen, in particular Cl.

According to a particular embodiment, the invention concerns the use of a compound of following formula (Ia):

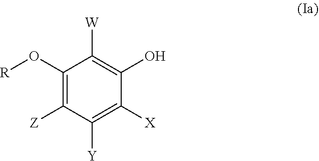

wherein:
R is a linear or branched alkyl in C₁ to C₄, optionally substituted by at least one halogen, R being in particular a methyl or a perhalogenoalkyl in C₁ to C₄;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
at least one of W, X, Y and Z representing a halogen.

According to a particular embodiment, at least two of W, X, Y and Z representing a halogen, in particular at least two of X, Y and Z representing a halogen.

According to a particular embodiment, X is chosen from the halogens, and at least one of Y and of Z is chosen from the halogens.

According to a particular embodiment, X and Z are each independently chosen from the halogens, Y representing in particular H or a halogen.

According to a particular embodiment X and Y are each independently chosen from the halogens, Z representing in particular H or a halogen.

According to a particular embodiment, said compound is not one of the following structures:

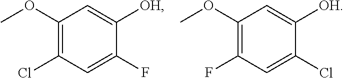

According to a particular embodiment, the invention concerns the use of a compound such as defined previously, provided that:
if X represents F and Y represents H, then Z is chosen from F, Br and I;
if X represents Cl and Y represents H, then Z is chosen from Cl, Br and I.

According to a particular embodiment, said compound is not one of the following structures:

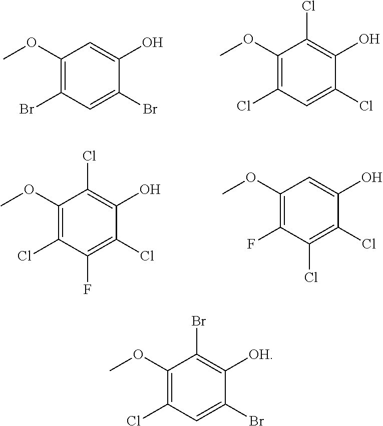

According to a particular embodiment, W is chosen from H and the linear or branched alkyls in C₁ to C₄, optionally substituted by at least one halogen, W being in particular H, a methyl or a perhalogenoalkyl in C₁ to C₄.

According to a particular embodiment, the invention concerns the use of a compound such as defined previously, provided that if Z represents F, then at least one of X and of Y is chosen from F, Br and I.

According to a particular embodiment, the invention concerns the use of a compound such as defined previously, provided that if Z represents Br, W and Y being in particular H, then X is chosen from F, Cl and I.

According to a particular embodiment, W is chosen from H and the linear or branched alkyls in C₁ to C₄, optionally substituted by at least one halogen, provided that:
if Z represents F, then at least one of X and of Y is chosen from F, Br and I; and
if Z represents Br, W and Y being in particular H, then X is chosen from F, Cl and I.

According to a particular embodiment, W represents H.

According to a particular embodiment, X, Y and Z are each independently chosen from the halogens.

According to a particular embodiment, the invention concerns the use of a compound such as defined previously, wherein:

W represents H; and
X, Y and Z are each independently chosen from the halogens; or
X and Z represent Br and Cl, X representing Br and Z representing Cl, or X representing Cl and Z representing Br, Y representing in particular H.

According to a particular embodiment, the invention concerns the use of a compound such as defined previously, wherein if Z represents F, then Y is chosen from Cl, Br and I.

According to a particular embodiment, the invention concerns the use of a compound such as defined previously, wherein:

W represents H; and
X, Y and Z are each independently chosen from the halogens; or
X and Z represent Br and Cl, X representing Br and Z representing Cl, or X representing Cl and Z representing Br, Y representing in particular H; or
X and Y represents a halogen, Z representing in particular H;
provided that:
if Z represents F, then at least one of X and of Y is chosen from F, Br and I; Y is chosen from Cl, Br and I.

According to a particular embodiment, said compound is chosen from the group formed by compounds of following structure:

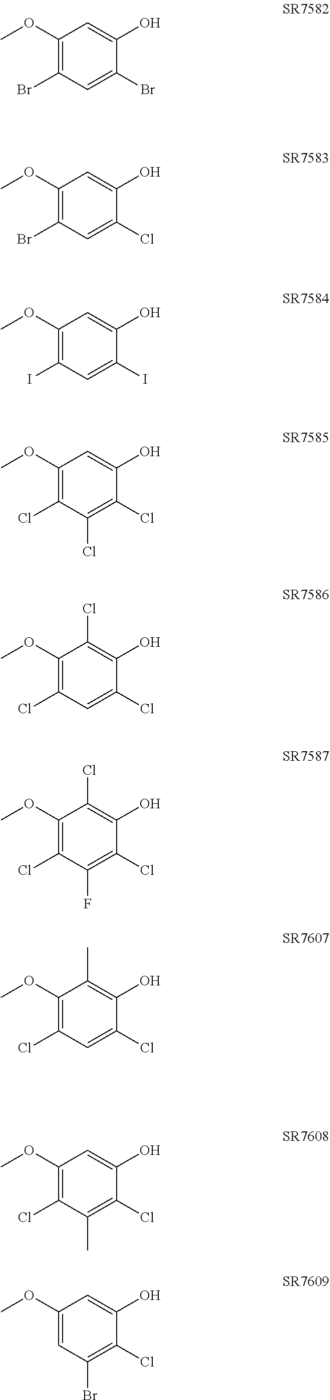

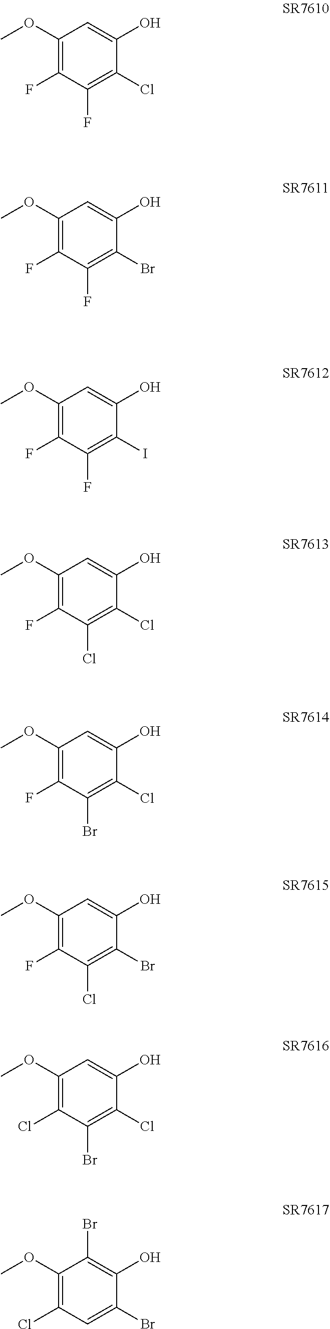

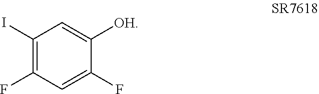

It should be noted that all the embodiments mentioned above regarding the compounds such as defined previously are applied individually or in combination.

According to a particular embodiment, the invention concerns the use of a compound such as defined previously as a pesticide, disinfectant of inert surfaces or preservative.

According to a particular embodiment, the invention concerns the use of a compound such as defined previously as an antimicrobial preservative.

According to a particular embodiment, the invention concerns the use of a compound such as defined previously as a bactericide or bacteriostatic, in particular as an antibacterial preservative.

The bacteria treated are in particular Gram+ or Gram−, for example Pseudomonas aeruginosa, Escherichia coli, Staphylococcus aureus, Streptococcus equi subsp zooepidemicus, Klebsiella pneumoniae or Stenotrophomonas maltophila, in particular Escherichia coli, Staphylococcus aureus, Streptococcus equi subsp zooepidemicus, Klebsiella pneumoniae or Stenotrophomonas maltophila.

The compound such as defined previously can be used as a bactericide or bacteriostatic active ingredient, in particular in soaps or antibacterial solutions.

The compound such as defined previously can also be used as preservative.

The compound such as defined previously is thus in particular a preservative of a composition, in particular a cosmetic composition, a pharmaceutical composition, a biological sample in particular of animal origin, or of a material, in particular a plastic material, for example for agribusiness, intended to kill or slow down the growth of microbes such as the bacteria, the mycetes, the viruses, or the parasites, in said composition or said material, but also in any material in contact with said composition or with said material.

Thus, the compounds of the invention can in particular be used as a preservative after impregnation or insertion into plastic materials, for example for the manufacture of food packaging, bin bags, sponges.

In particular, the compounds of the invention, for example the compound sr7615, can be used as biocide, in particular preservative, in a layer of weldable plastic material, in particular included in an animal semen packaging bag.

The cosmetic compositions are in particular soaps, shower gels, toothpastes, mouthwashes, moisturisers, etc.

According to a particular embodiment, the invention concerns the use of a compound such as defined previously for disinfecting a fluid or a surface, in particular water, air, floors, swimming pools, worksurfaces, toilets by reducing, in particular, the number of viable bacterial cells, bacterial cells being in particular Gram− or Gram+, in particular multi-resistant to biocides and/or antibiotics, bacterial cells being, for example, Pseudomonas aeruginosa, Escherichia coli, Staphylococcus aureus, Streptococcus equi subsp zooepidemicus, Klebsiella pneumoniae or Stenotrophomonas maltophila, in particular Escherichia coli, Staphylococcus aureus, Streptococcus equi subsp zooepidemicus, Klebsiella pneumoniae or Stenotrophomonas maltophila.

According to a particular embodiment, the compound such as defined previously, optionally used in the presence of water, is applied onto the fluid or the surface by spraying, vaporisation, soaking, staining, fumigation, electrostatic spraying, or, for liquids only, by dissolution.

Surfaces, in particular in communities, in a hospital environment or in agribusiness production facilities, in particular floors, worksurfaces, toilets, facilities such as spas, swimming pools, cooling towers, agribusiness production facilities, hospital environments, are likely to be disinfected by a compound such as described previously.

According to a particular embodiment, the invention concerns the use of a compound such as defined previously, as an antimicrobial, in particular as an antibacterial, in humans or in animals, said use being performed in particular topically.

According to a particular embodiment, the invention concerns a compound of formula (I) or (Ia) such as defined previously, for its use as an antimicrobial, in particular as an antibacterial, in humans or in animals, said use being performed in particular topically.

According to a particular embodiment, the invention concerns a pharmaceutical composition comprising as an active substance, a compound of formula (I) or (Ia) such as defined previously and a pharmaceutically acceptable excipient, for its use as an antimicrobial, in particular as an antibacterial, in humans or in animals, said use being performed in particular topically.

According to another aspect, the invention concerns a compound of following formula (II):

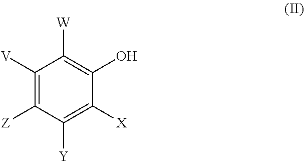

wherein:
V represents:
a group —OR wherein R is a linear or branched alkyl in C₁ to C₄, optionally substituted by at least one halogen, R being in particular a methyl or a perhalogenoalkyl in C₁ to C₄; or
a halogen;
   W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
   provided that at least two of W, X, Y and Z represent a halogen;
   as well as its pharmaceutically acceptable salts;
   said compound not being one of the following formulae:

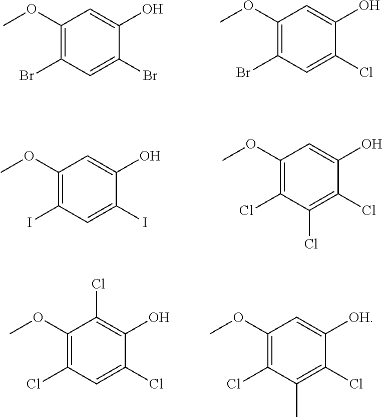

According to a particular embodiment, V represents a group —OR such as defined previously, or I.

According to a particular embodiment, V represents I.

According to a particular embodiment, the invention concerns a compound of following formula (II):

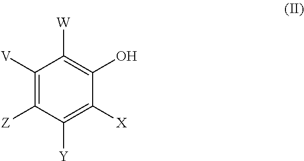

wherein:
V represents:
a group —OR, wherein R is a linear or branched alkyl in C₁ to C₄, optionally substituted by at least one halogen, R being in particular a methyl or a perhalogenoalkyl in C₁ to C₄; or
I;
   W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
   provided that at least two of W, X, Y and Z representing a halogen;
   as well as its pharmaceutically acceptable salts;
   said compound not being one of the following formulae:

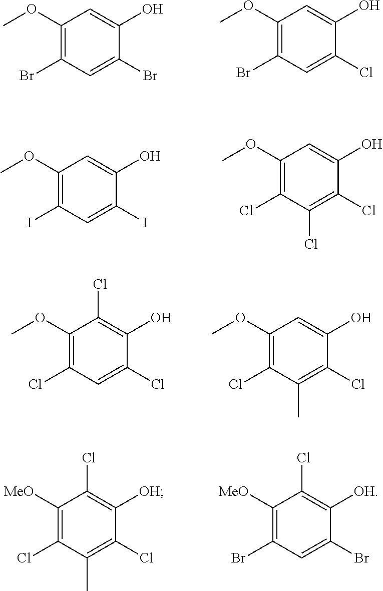

According to a particular embodiment, W does not represent a halogen, in particular Cl, and Y does not represent a linear or branched alkyl in C₁ to C₄, in particular Me.

According to a particular embodiment, when Y represents a linear or branched alkyl in C₁ to C₄, in particular Me, then W, X and Z do not represent a halogen, in particular Cl.

According to a particular embodiment, when Y represents a linear or branched alkyl in C₁ to C₄, in particular Me, then X and Z do not represent a halogen, in particular Cl.

According to a particular embodiment, when Y represents H, then W, X and Z do not represent a halogen, in particular Br or Cl.

According to a particular embodiment, the invention concerns a compound of following formula (IIa):

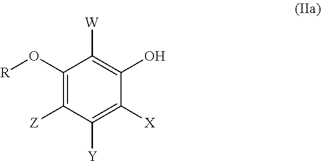

wherein:
R is a linear or branched alkyl in C₁ to C₄, optionally substituted by at least one halogen, R being in particular a methyl or a perhalogenoalkyl in C₁ to C₄;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
provided that:
X, Y and Z are each independently chosen from the halogens; or
at least one of W and Y, in particular W, does not represent H;
   said compound not being one of the following formulae:

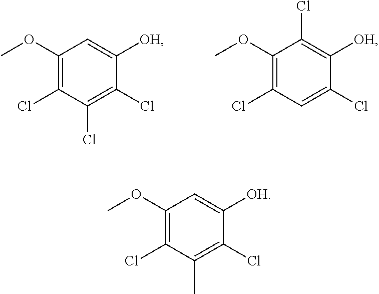

According to a particular embodiment, the invention concerns a compound of following formula (IIa):

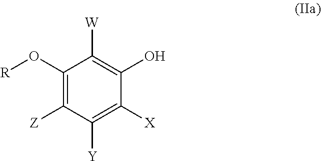

wherein:
R is a linear or branched alkyl in C₁ to C₄, optionally substituted by at least one halogen, R being in particular a methyl or a perhalogenoalkyl in C₁ to C₄;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
provided that:
X, Y and Z are each independently chosen from the halogens; or
at least one of W and Y, in particular W, does not represent H;
   said compound not being one of the following formulae:

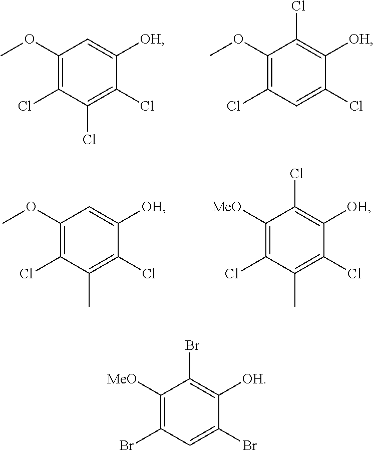

According to a particular embodiment, Y is chosen from H and the halogens; said compound not being one of the following formulae:

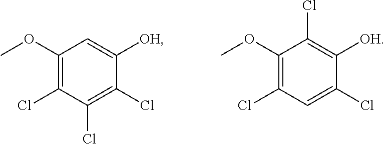

It should be noted that all the embodiments mentioned above regarding compounds of formula (I), (Ia), (II) or (IIa) such as defined previously, can also be applied here, individually or in combination.

According to another aspect, the invention concerns a pharmaceutical composition comprising as active substance, a compound of formula (II) or (IIa) such as defined previously and a pharmaceutically acceptable excipient.

It should be noted that all the embodiments mentioned above regarding compounds of formula (I), (Ia), (II) or (IIa) such as defined previously, can also be applied here, individually or in combination.

For the preparation of pharmaceutical compositions from the compounds of the present invention, pharmaceutically acceptable excipients can be solid or liquid. The preparations in solid form comprise powders, tablets, dispersible granules, capsules and suppositories.

Compositions in liquid form comprise solutions, suspensions and emulsions. For example, sterile water, alcohols such as for example, ethanol, polyethylene glycol and/or propylene glycol can be suitable for preparing such liquid forms.

According to another aspect, the present invention also relates to the pharmaceutically acceptable salts of the compounds described previously. “Pharmaceutically acceptable salts” comprise, in particular, the salts of compounds of the present invention which are derivatives of the combination of these compounds with non-toxic acids.

These acids comprise inorganic acids such as hydrochloric, hydrobromic, hydroiodic, sulphuric, nitric and phosphoric acid, as well as organic acids such as acetic, citric, propionic, tartaric, glutamic, salicylic, oxalic, methanesulfonic, para-toluenesulfonic, succinic and benzoic acid.

In addition to pharmaceutically acceptable salts, other salts are included in the invention. They can be used as intermediaries in the purification of compounds, in the preparation of other salts or in the identification and the characterisation of compounds or intermediaries.

Synthesis

The compounds of the present invention can be prepared according to methods well-known to the person skilled in the art, including, but without being limited to them, those which are described below, or by modifications of these methods by applying standard techniques known to the person skilled in the art of organic synthesis. Suitable modifications and substitutions will be well-known or easily accessible, from reading scientific literature, to the person skilled in the art. In particular, such methods can be found in R. C. Larock, Comprehensive Organic Transformations, Wiley-VCH Publishers, 1999.

All of the methods disclosed in association with the present invention can be applied on any scale, including milligrams, grams, multigrams, kilograms, multikilograms, or on a commercial industrial scale.

The compounds of the present invention can be prepared by various synthetic methods. Reagents and starting materials are commercially available, or easily synthesised by techniques well-known to a person skilled in the art. All of the substitutes, unless otherwise specified, are such as defined previously.

In the reactions described below, it can be necessary to protect the reactive functional groups, for example hydroxy groups, when these are desired in the end product, in order to prevent their undesired participation in the reactions. Conventional protective groups can be used according to common practice, for example, see T. W. Greene and P. G. M. Wuts in Protective Groups in Organic Chemistry, 3^{rd} ed., John Wiley and Sons, 1999; J. F. W. McOmie in Protective Groups in Organic Chemistry, Plenum Press, 1973.

The general methods for preparing the compounds of the present invention are presented in the schemes below.

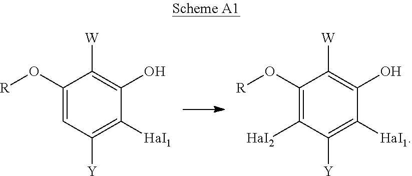

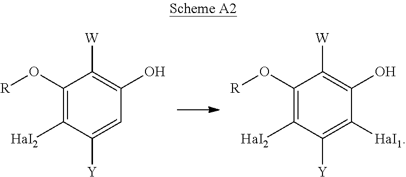

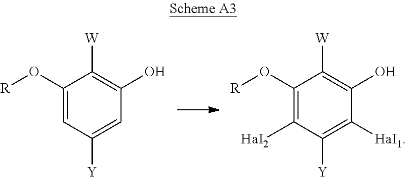

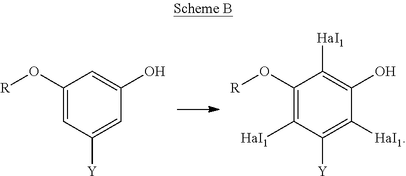

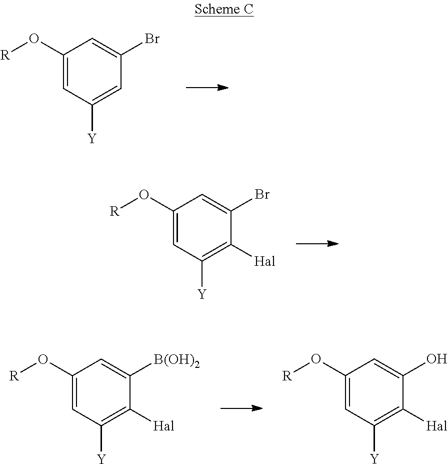

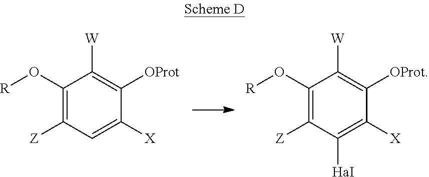

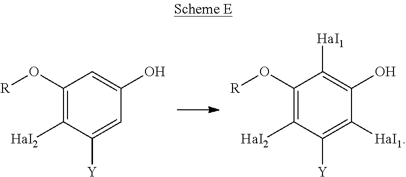

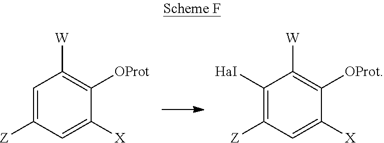

Concerning the schemes A1-A3, B, C, E and F, the halogenation reaction can be, for example, achieved using n-halogenosuccinimide (in particular, n-bromosuccinimide, n-chlorosuccinimide, n-iodosuccinimide), for example in a solvent such as acetonitrile, acetic acid or trifluoroacetic acid (TFA), at a temperature in particular comprised of 20° C. at reflux.

The group Prot is an ad hoc protective group of hydroxyls, such as mentioned above.

Regarding scheme C, the substitution of —Br by the group —OH can be performed by boration using a strong base of type nBuLi and of B(OiPr)₃ then treatment with oxygenated water.

Relative to the scheme D, the reaction presented can be achieved by deprotonation, using nBuLi for example, then halogenation, using in particular C₂Cl₆ or CBr₄.

### Definitions

As used in the present description, the term “about” refers to the interval of values of 10% of a specific value. As an example, the expression “about 120 mg” comprises values of 120 mg±10%, i.e. the values of 108 mg to 132 mg.

In the sense of the present description, the percentages refer to percentages by weight with respect to the total weight of the formulation, unless otherwise indicated.

In this sense, the ranges of values in the form of “x-y” or “from x to y” or “between x and y” include the limits x and y, as well as integers comprised between these limits. As an example, “1-5”, or “from 1 to 5” or “between 1 and 5” denote the integers 1, 2, 3, 4 and 5. The preferred embodiments include each integer taken individually in the value range, as well as any sub-combination of these integers. As an example, the preferred values for “1-5” can comprise the integers 1, 2, 3, 4, 5, 1-2, 1-3, 1-4, 1-5, 2-3, 2-4, 2-5, etc.

“Halogen” denotes, unless otherwise indicated, any element in column VIIB of the periodic table. This includes in particular F, Cl, Br, I, or any combination of two, three, or four of these halogens.

According to the present invention, “alkyl” radicals represent saturated hydrocarbon radicals, in a straight or branched chain, from 1 to 8 carbon atoms, in particular from 1 to 6 carbon atoms, preferably from 1 to 4 carbon atoms. When they are linear, they can be in particular methyl, ethyl, propyl, butyl, pentyl, hexyl radicals. When they are branched, they can be in particular isopropyl, tert-butyl, 2-methylbutyl, 2-methylpentyl, and 1-methylpentyl radicals.

“Perhalogenoalkyl” denotes an alkyl such as defined above, wherein all of the hydrogen atoms are replaced by halogen atoms.

“Biocide” denotes pesticides, as well as antimicrobials for medical, veterinary, domestic or industrial use, and disinfectants of fluids and of surfaces, in particular water, air, floors, swimming pools, worksurfaces, toilets, etc.

“Surface”, unless otherwise indicated, denotes in particular the surfaces of living tissues, in particular the surface of plants (for example, leaf surfaces), and skin (on humans or animals), as well as inert surfaces, in particular organic or inorganic inert surfaces, for example floors and worksurfaces.

In the case of pesticides, the surface considered is in particular that of a plant.

In the case of antimicrobials, the surface considered is in particular that of skin, on humans or on animals.

In the case of disinfectants, the surface considered is in particular an inert surface.

“Antimicrobial” denotes an active substance which kills or slows down the growth of microbes such as the bacteria, the mycetes, the viruses, or the parasites, in particular in humans or animals.

“Antimicrobial” or “bactericide” denotes an active substance which kills or slows down the growth of bacteria, in particular in humans or animals.

“Antimicrobial preservative” denotes an additive of a composition, in particular a cosmetic composition, a pharmaceutical composition, or a material, in particular a plastic material, intended to kill or slow down the growth of microbes such as the bacteria, the mycetes, the viruses, or the parasites in said composition or said material, but also in any material in contact with said composition or with said material.

“Antibacterial preservative” denotes an additive of a composition, in particular a cosmetic composition, a pharmaceutical composition, or a material, in particular a plastic material, intended to kill or slow down the growth of bacteria in said composition or said material, but also in any material in contact with said composition or with said material.

“Disinfectant” denotes a product which kills or inactivates microorganisms, such as the bacteria, viruses and protozoas on inert surfaces or within fluids such as water and air.

As used here, the term “pharmaceutically acceptable” refers to compounds, compositions and/or dosing forms which are, in the scope of a valid medical judgement, adapted for use in contact with inferior human and animal cells without toxicity, irritation, undue allergic response and similar, and are proportionate to a reasonable benefit/risk ratio.

### EXAMPLES

### Example 1: Synthesis of Compounds of the Invention

### Synthesis of the Compound SR 7580:

This compound was obtained by following the operating method such as illustrated in the scheme below:

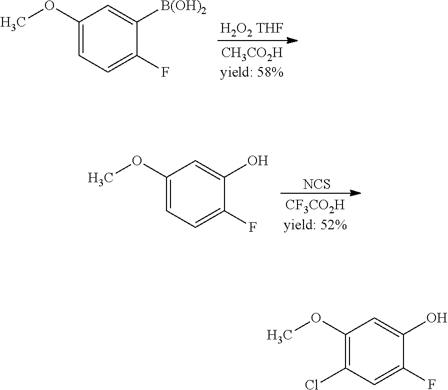

NMR ¹H (δ PPM) CDCl₃: 7.10 (d, J=9.8, 1H), 6.60 (d, J=7.8, 1H), 5.52 (s, 1H), 3.82 (s, 3H).

### Synthesis of the Compound SR 7581:

This compound was obtained by following the operating method such as illustrated in the scheme below:

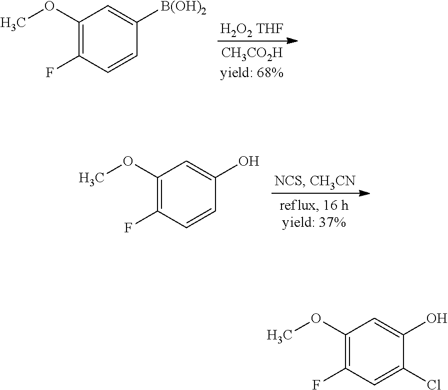

NMR ¹H (δ PPM) CDCl₃: 7.06 (d, J=10.5 1H), 6.65 (d, J=7.8, 1H), 5.32 (s, 1H), 3.84 (s, 3H).

### Synthesis of the Compound SR 7582:

This compound was obtained by following the operating method such as illustrated in the scheme below:

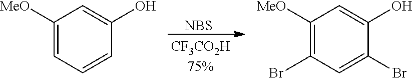

NMR ¹H (δ PPM) CDCl₃: 7.50 (s, 1H), 6.54 (s, 1H), 5.44 (s, 1H), 3.78 (s, 3H).

### Synthesis of the Compound SR 7583:

This compound was obtained by following the operating method such as illustrated in the scheme below:

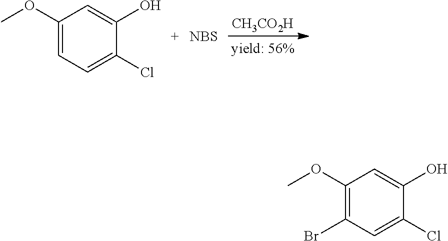

### Synthesis of the Compound SR 7584:

This compound was obtained by following the operating method such as illustrated in the scheme below:

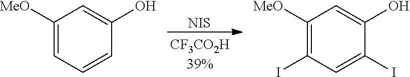

NMR ¹H (δ PPM) CDCl₃: 7.90 (s, 1H), 6.53 (s, 1H), 5.27 (s, 1H), 3.83 (s, 3H).

### Synthesis of the Compound SR 7585:

This compound was obtained by following the operating method such as illustrated in the scheme below:

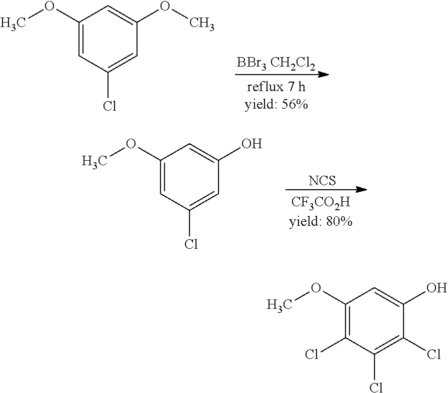

NMR ¹H (δ PPM) CDCl₃: 6.59 (s, 1H), 5.65 (s, 1H), 3.86 (s, 3H).

### Synthesis of the Compound SR 7586:

This compound was obtained by following the operating method such as illustrated in the scheme below:

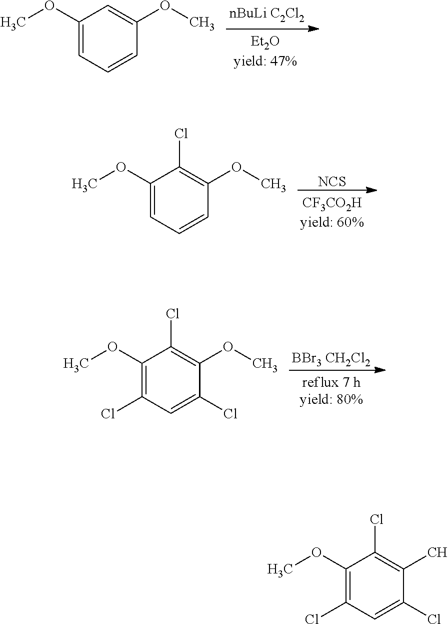

NMR ¹H (δ PPM) CDCl₃: 7.32 (s, 1H), 5.85 (s, 1H), 3.83 (s, 3H).

### Synthesis of the Compound SR 7587:

This compound was obtained by following the operating method such as illustrated in the scheme below:

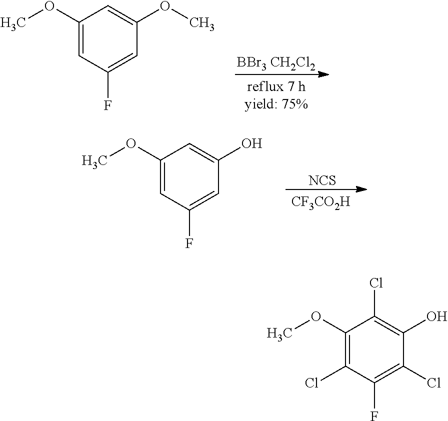

To a solution of 0.5 g of 3-fluoro-5-methoxyphenol in 10 ml of TFA, 1.46 g of NCS (3.1 eq.) is added. The reaction mixture is stirred for 48 hours at 25° C. Then, water and ice are added. A precipitation is formed, and it is filtered on a frit and then rinsed with water. We extract using AcOEt. Silica gel is purified by chromatography on a column (eluant: CH₂Cl₂). 0.52 g is obtained, yield=60%. FP=104° C. (white solid).

NMR ¹³C (δ ppm) CDCl₃: 155.0 and 152.5 (C—F), 152.5 (d, C—O), 147.9 (d, C—O), 111.95 (d, C—Cl), 109.6 (d, C—Cl), 106.1 (d, C—Cl).

### Synthesis of the Compound SR 7607:

This compound was obtained by following the operating method such as illustrated in the scheme below:

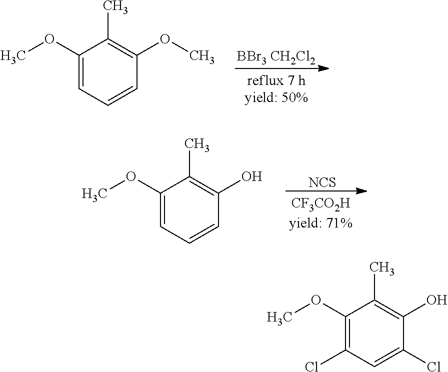

To a solution of 5.2 g of 2-methyl-3-methoxyphenol in 20 ml of TFA, 11.6 g of NCS (2.2 eq.) are added. The reaction mixture is stirred for 16 hours at A.T. 1N NaOH is added, washed by CH₂Cl₂. The aqueous phase is acidified by 3N HCl and extracted using CH₂Cl₂. 5.5 g of product is obtained. Yield=71%. FP=55° C. (white solid).

NMR ¹H (δ ppm) CDCl₃: 7.19 (s, 1H, H₃), 5.54 (s, 1H, OH), 3.77 (s, 3H, OCH₃), 2.15 (s, 3H, CH₃).

### Synthesis of the Compound SR 7608:

This compound was obtained by following the operating method such as illustrated in the scheme below:

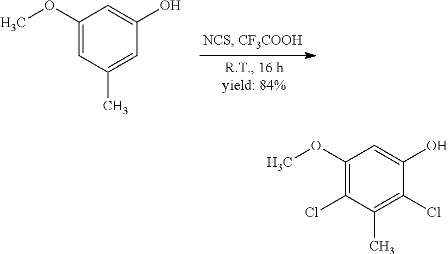

### Synthesis of the Compound SR 7609:

This compound was obtained by following the operating method such as illustrated in the scheme below:

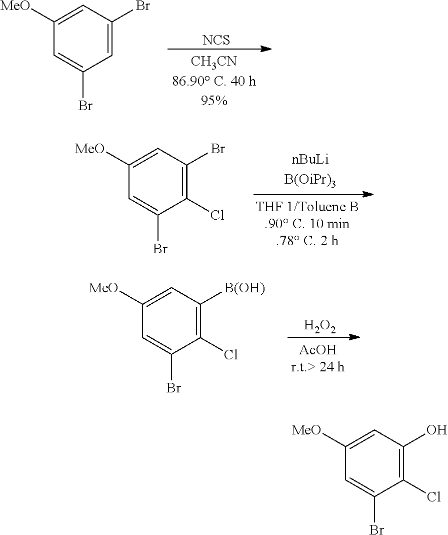

NMR ¹H (δ PPM) CDCl₃: 6.79 (d, J=2.8, 1H), 6.56 (d, J=2.8, 1H), 5.63 (s, 1H), 3.77 (s, 3H).

### Synthesis of the Compound SR 7610:

This compound was obtained by following the operating method such as illustrated in the scheme below:

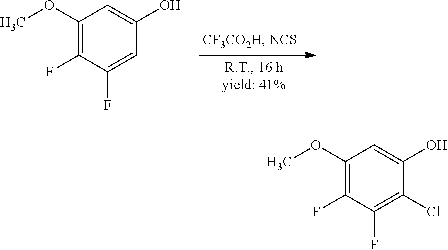

To a solution of 3.4 g of 3,4-difluoro-5-methoxyphenol in 20 ml of TFA, 2.98 g of NCS (1.05 eq.) are added in small quantities (the flask is immersed in an ice bath during the addition). It is left to rise to room temperature and the reaction mixture is stirred for 16 hours. Then, water and ice are added. A precipitation is formed, and it is filtered on a frit and then rinsed with water. The residue is returned into CH₂Cl₂ and washed with water. The organic phase is dried on MgSO₄, filtered and evaporated. Purification is carried out by chromatography on a silica gel column, with as eluent: CH₂Cl₂/AcOEt/cyclohexane=3/1/1. 1.7 g of product is obtained.

Yield=41%. FP=87.6° C. (white solid).

NMR ¹H (δ ppm) CDCl₃ 6.48 (dd, 1H), 5.40 (s, 1H, OH), 3.87 (s, 3H).

### Synthesis of the Compound SR 7611:

This compound was obtained by following the operating method such as illustrated in the scheme below:

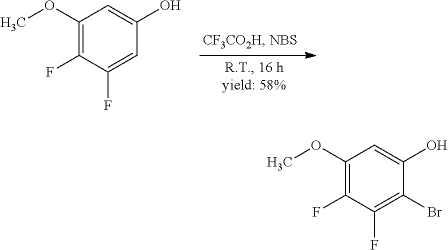

To a solution of 0.23 g of 3,4-difluoro-5-methoxyphenol in 4 ml of TFA, 0.27 g of NBS (1.05 eq.) is added in small quantities (the flask is immersed in an ice bath during the addition). It is left to rise to room temperature and the reaction mixture is stirred for 16 hours. Then, water and ice are added. A precipitation forms itself and it is filtered on a frit then rinsed with water. The residue is taken up in CH₂Cl₂ and washed with water. The organic phase is dried on MgSO₄, filtered and evaporated. Purification is carried out by chromatography on a silica gel column, with, as eluent: CH₂Cl₂. 0.2 g of product is obtained. Yield=58%. FP=67.5° C. (white solid).

NMR ¹H (δ ppm) CDCl₃ 6.50 (dd, 1H), 5.48 (s, 1H, OH), 3.87 (s, 3H).

### Synthesis of the Compound SR 7612:

This compound was obtained by following the operating method such as illustrated in the scheme below:

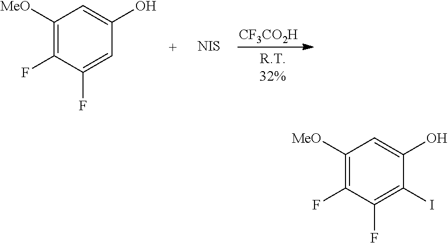

To a solution of 0.28 g of 3,4-difluoro-5-methoxyphenol in 5 ml of TFA, 0.41 g of NIS (1.05 eq.) is added in small quantities (the flask is immersed in an ice bath during the addition). It is left to rise to room temperature and the reaction mixture is stirred for 16 hours. Then, water and ice are added. A precipitation forms itself and it is filtered on a frit then rinsed with water. The residue is returned into CH₂Cl₂ and washed with water. The organic phase is dried on MgSO₄, filtered and evaporated. Purification is carried out by chromatography on a silica gel column, with, as eluent: CH₂Cl₂. 0.16 g of product is obtained. Yield=32%. FP=91.1° C. (brown solid).

NMR ¹H (δ ppm) CDCl₃ 6.52 (dd, 1H), 5.36 (s, 1H, OH), 3.87 (s, 3H).

### Synthesis of the Compound SR 7613:

This compound was obtained by following the operating method such as illustrated in the scheme below:

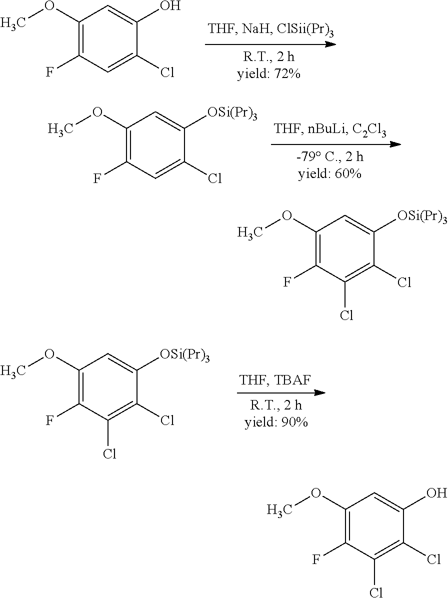

To a solution of 4.18 g of 4-fluoro-5-methoxyphenol in 100 ml of 1,2-dichloroethane, 4.1 g of NCS (1.05 eq.) is added. The reaction mixture is refluxed for 4, 5 days (progression of the reaction controlled by LCMS). It is extracted using CH₂Cl₂. Purification is carried out by chromatography on a silica gel column (cyclohexane/AcOEt=1/1). Yield=87%.

NMR ¹H (δ ppm) CDCl₃: 7.06 (d, 1H, J=10.5 Hz, H₆), 6.65 (d, 1H, J=7.8 Hz, H₆), 5.32 (s, 1H, OH), 3.84 (s, 3H, OCH₃).

To a solution of 4.7 g of 2-chloro-4-fluoro-5-methoxyphenol in 100 ml of THF at 0° C., 2.1 g of NaH (2 eq.) are added in small quantities. It is left to rise to room temperature and the reaction mixture is stirred for 6 hours. Then, 7.4 ml of ClSiiPr₃ (1.3 eq.) is added at 0° C. After 16 hours of stirring, it is hydrolysed by water. It is extracted thanks to AcOEt and purified by chromatography on a silica gel column (cyclohexane/CH₂Cl₂=1/1). Yield=64%.

NMR ¹H (δ ppm) CDCl₃: 7.09 (d, J=10.8 Hz, 1H, H₆), 6.55 (d, J=8 Hz, 1H, H₃), 3.82 (s, 3H, OCH₃), 1.2 (m, 3H, 3H_{iPr}), 1.13 (d, 18H, 6CH₃).

0.45 g of (2-chloro-4-fluoro-5-methoxyphenoxy)triisopropylsilane and 70 ml of THF are introduced into a three-necked flask under mechanical stirring and under nitrogen atmosphere at −70° C., 1.4 ml of nBuLi (2.5M 2.5 eq.) are added with caution. The solution is left to stir for 1 hour then 0.8 g of C₂Cl₆ (2.5 eq.) is added into the solution in the THF at −70° C.

After 1 hour of stirring, the reaction mixture is gently raised to room temperature. It is extracted using AcOEt and the residue obtained is purified by chromatography on a silica gel column (cyclohexane then cyclohexane/CH₂Cl₂=1/1). 0.3 g of product is obtained, Yield=60%.

NMR ¹H (δ ppm) CDCl₃: 6.50 (d, J=7.6 Hz, 1H, H₆), 3.83 (s, 3H, OCH₃), 1.30 (m, 3H, 3H_{iPr}), 1.12 (d, 18H, 6CH₃).

To a solution of 10 g of 2,3-dichloro-4-fluoro-5-methoxyphenoxy)triisopropylsilane in 100 ml of THF, 35.4 ml of TBAF (1M, 1.3 eq.) are added. The reaction mixture is stirred for 72 hours at room temperature. It is extracted using AcOEt and purified by chromatography on a silica gel column (CH₂Cl₂). 5.2 g of product are obtained, Yield=91%. FP=100.1° C. (beige solid).

NMR ¹H (δ ppm) CDCl₃: 6.61 (d, J=7.2 Hz, 1H, H₆), 5.51 (s, 1H, OH), 3.86 (s, 3H, OCH₃).

### Synthesis of the Compound SR 7614:

This compound was obtained by following the operating method such as illustrated in the scheme below:

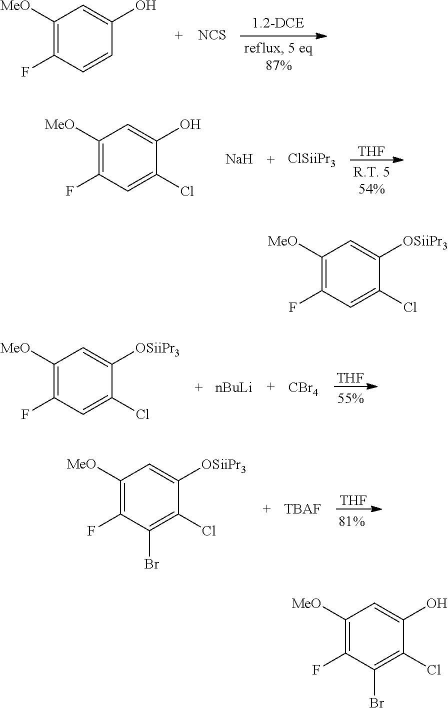

To a solution of 4.18 g of 4-fluoro-5-methoxyphenol in 100 ml of 1,2-dichloroethane, 4.1 g of NCS (1.05 eq.) are added. The reaction mixture is refluxed for 4.5 days (progression of the reaction controlled by LCMS). It is extracted using CH₂Cl₂. Purification is carried out by chromatography on a silica gel column (cyclohexane/AcOEt=1/1). Yield=87%.

NMR ¹H (δ ppm) CDCl₃: 7.06 (d, 1H, J=10.5 Hz, H₆), 6.65 (d, 1H, J=7.8 Hz, H₆), 5.32 (s, 1H, OH), 3.84 (s, 3H, OCH₃).

To a solution of 4.7 g of 2-chloro-4-fluoro-5-methoxyphenol in 100 ml of THF at 0° C., 2.1 g of NaH (2 eq.) are added in small quantities. It is left to rise to room temperature and the reaction mixture is stirred for 6 hours. Then, 7.4 ml of ClSiiPr₃ (1.3 eq.) are added at 0° C. After 16 hours of stirring, it is hydrolysed by water. It is extracted using AcOEt and purified by chromatography on a silica column (cyclohexane/CH₂Cl₂=1/1). Yield=64%.

NMR ¹H (δ ppm) CDCl₃: 7.09 (d, J=10.8 Hz, 1H, H₆), 6.55 (d, J=8 Hz, 1H, H₃), 3.82 (s, 3H, OCH₃), 1.2 (m, 3H, 3H_{iPr}), 1.13 (d, 18H, 6CH₃).

0.59 g of (2-chloro-4-fluoro-5-methoxyphenoxy)triisopropylsilane and 70 ml of THF are introduced in a three-necked flask under mechanical stirring and nitrogen atmosphere at −70° C., 1.8 ml of nBuLi (2.5M 2.5 eq.) are added with caution. The solution is left under stirring for 1 hour then 1.5 g of CBr₄ (2.5 eq.) are added into solution in the THF at −78° C. After 30 minutes of stirring, the reaction mixture is gently raised to room temperature. It is extracted using AcOEt and purified by chromatography on a silica gel column (cyclohexane then cyclohexane/CH₂Cl₂=1/1). Yield=55%.

NMR ¹H (δ ppm) CDCl₃: 6.56 (d, J=8 Hz, 1H, H₆), 3.83 (s, 3H, OCH₃), 1.28 (m, 3H, 3H_{iPr}), 1.13 (d, 18H, 6CH₃).

To a solution of 0.4 g of 3-bromo-2-chloro-4-fluoro-5-methoxyphenoxy)triisopropylsilane in 50 ml of THF, 1.3 ml of TBAF (1M, 1.3 eq.) are added. The reaction mixture is stirred for 16 hours at room temperature. It is extracted using AcOEt and purified by chromatography on a silica gel column (CH₂Cl₂). Yield=81%. FP=110° C. (beige solid).

NMR ¹H (δ ppm) CDCl₃: 6.68 (dd, J=7.6 Hz, J=1.2 Hz, H₆), 5.47 (s, 1H, OH), 3.87 (s, 3H, OCH₃).

### Synthesis of the Compound SR 7615:

This compound was obtained by following the operating method such as illustrated in the scheme below:

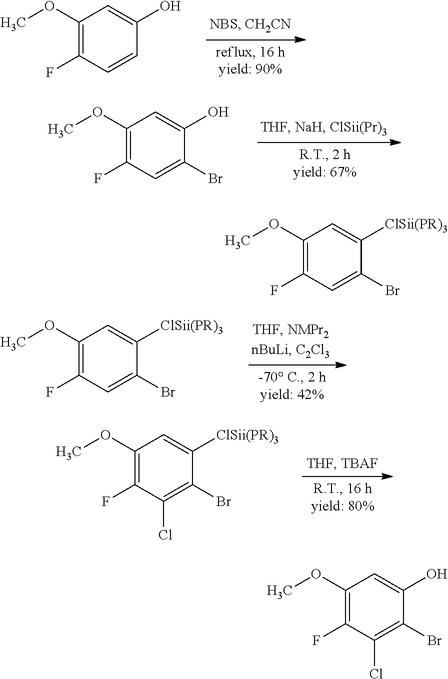

To a solution of 9.14 g of 4-fluoro-5-methoxyphenol in 150 ml of CH₃CN, 12.02 g of NBS (1.05 eq.) are added. The reaction mixture is refluxed for 16 hours. It is extracted using AcOEt and the obtained residue is purified by chromatography on a silica gel column in (CH₂Cl₂). Yield=90%.

NMR ¹H (δ ppm) CDCl₃: 7.19 (d, J=10 Hz, 1H, H₃), 6.68 (d, J=7.6 Hz, 1H, H₆), 5.31 (s, 1H, OH), 3.85 (s, 3H, OCH₃).

To a solution of 3.07 g of 2-bromo-4-fluoro-5-methoxyphenol in 100 ml of THF at 0° C., 1.1 g of NaH (2 eq.) are added in small quantities. It is left to rise to room temperature and the reaction mixture is stirred for 3 hours. Then, 3.6 ml of ClSiiPr₃ (1.2 eq.) are added at 0° C. After 24 hours of stirring, it is hydrolysed by water. It is extracted with AcOEt and purified by chromatography on a silica column (cyclohexane/CH₂Cl₂=1/1). Yield=67%.

NMR ¹H (δ ppm) CDCl₃: 7.23 (d, J=10.4 Hz, 1H, H₃), 6.55 (d, J=8 Hz, 1H, H₆), 3.82 (s, 1H, OCH₃), 1.31 (m, 3H, 3H_{iPr}), 1.14 (d, 18H, 6CH₃).

4.1 ml of HNiPr₂ (2.5 eq.) and 180 ml of THF are introduced in a three-necked flask under mechanical stirring and under nitrogen atmosphere, between −10° C. to 0° C., 11.7 ml of nBuLi (2.5M 2.5 eq.) are added with caution. The solution is left under stirring for 30 minutes, then 4.4 g of (2-bromo-4-fluoro-5-methoxy-phenoxy)-triisopropylsilane in solution in the THF are added at −70° C. After 1 hours of stirring, 6.9 g of C₂Cl₆ (2.5 eq.) are added in solution in the THF. The reaction mixture is stirred for 1 hour, then gently raised to room temperature and left under stirring for 1 hour. It is extracted using AcOEt and purified by chromatography on a silica column (cyclohexane/CH₂Cl₂=1/1). Yield=94%.

NMR ¹H (δ ppm) CDCl₃: 6.51 (d, J=7.4 Hz, H₆), 3.83 (s, 3H, OCH₃), 1.31 (m, 3H, 3H_{iPr}), 1.14 (d, 18H, 6CH₃).

To a solution of 1 g of 2-bromo-3-chloro-4-fluoro-5-methoxyphenoxy)triisopropylsilane in 100 ml of THF, 3.2 ml of TBAF (1M, 1.3 eq.) are added. The reaction mixture is stirred for 16 hours at room temperature. It is extracted using AcOEt and purified by chromatography on a silica gel column (cyclohexane/CH₂Cl₂=1/1).

Yield=80%. FP=104° C. (brown solid).

NMR ¹H (δ ppm) CDCl₃: 6.65 (d, J=7.2 Hz, H₆), 5.45 (s, 1H, OH), 3.87 (s, 3H, OCH₃).

### Synthesis of the Compound SR 7616:

This compound was obtained by following the operating method such as illustrated in the scheme below:

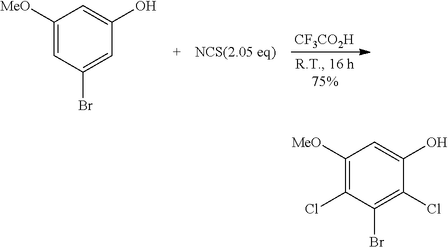 [SMILES: COc1cc(O)cc(Br)c1 ; "CF3CO2H" ; COc1cc(O)c(Cl)c(Br)c1Cl]

To a solution of 0.4 g of 3-bromo-5-methoxyphenol in 5 ml of TFA, 0.54 g of NCS (2.05 eq.) is added in small quantities (the flask is immersed in an ice bath during the addition). It is left to rise to room temperature and the reaction mixture is stirred for 16 hours. Then, 50 ml of water and ice are added. A precipitation is formed, and it is filtered on a frit then rinsed with water. The residue is taken-up in CH₂Cl₂ and washed with water. The organic phase is dried on MgSO₄, filtered and evaporated. Purification is carried out by chromatography on a silica gel column with eluant CH₂Cl₂. 0.4 g (Yield=75%) of expected product is obtained.

FP=136° C. (white solid)

NMR ¹H (δ ppm) CDCl₃: 6.66 (s, 1H, H₆), 5.66 (s, 1H, OH), 3.88 (s, 3H, OCH₃).

### Synthesis of the Compound SR 7617:

This compound was obtained by following the operating method such as illustrated in the scheme below:

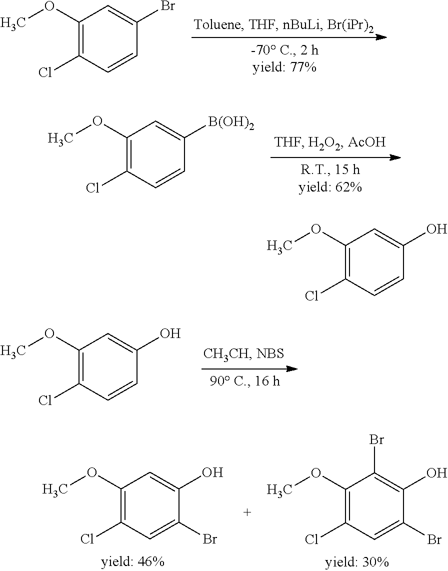

To a solution of 5.8 g of 4-chloro-3-methoxyphenol in 50 ml of CH₃CN, 7.8 g of NBS (1.2 eq.) are added. The reaction mixture is brought to reflux for 16 hours, 0.8 g of NBS are added, and the mixture is brought to reflux for 4 hours. It is extracted by AcOEt and purified by chromatography on a silica gel column (cyclohexane/AcOEt=2/1). 4 g of 2-bromo-4-chloro-5-methoxyphenol (Yield=46%) and 3.5 g of 2,6-dibromo-4-chloro-5-methoxyphenol (Yield=30%-sr7617) are obtained.

2-bromo-4-chloro-5-methoxyphenol: NMR ¹H (δ ppm) CDCl₃: 7.43 (s, 1H, H₃), 6.64 (s, 1H, H₆), 5.54 (s, 1H, OH), 3.89 (s, 3H, OCH₃).

SR 7617: FP=96.6° C. (white solid). NMR ¹H (δ ppm) CDCl₃: 7.52 (s, 1H, H₃), 5.89 (s, 1H, OH), 3.89 (s, 3H, OCH₃).

### Synthesis of the Compound SR 7618:

This compound was obtained by following the operating method such as illustrated in the scheme below:

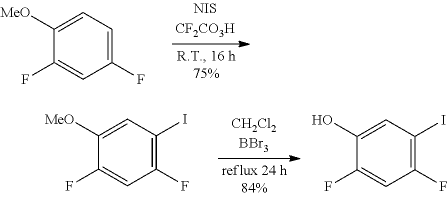

NMR ¹H (δ PPM) CDCl₃: 7.27 (dd, J=6.1, J=9.3 1H), 6.87 (dd, J=7.1, J=10.2, 1H), 5.12 (s, 1H).

### Biological Evaluations

Each test was controlled with three controls (T):
1 T culture positive (single strain)
1 T culture negative (strain in the presence of triclosan)
1 T DMSO (strain in the presence of DMSO)

The bacterial growth is evaluated by measuring the optical density (DO) at 600 nm using NanoDrop.

To compare the effect of the molecule tested with that of triclosan, the following ratio is calculated: [DO(single strain)−DO(strain+triclosan)]/[DO(single strain)−DO(strain+molecule)].

This ratio permits a classification of the molecules in 4 effect levels:
0=no effect observed;
1=effect observed only when the molecule is tested pure;
2=effect observed at several concentrations, but which gradually decreases the dilutions;
3=strong effect observed at each concentration of molecule tested.

### Example 2: Evaluation of Compounds of the Invention on a Micrococcus luteus Reference Strain

The molecules are put in a solution in DMSO and diluted in PBS.

The results are outlined in the table below:

| Tested molecules | Effect level |
|---|---|
| sr7583 | 3 |
| sr7584 | 2 |
| sr7585 | 3 |
| sr7607 | 2 |
| sr7608 | 2 |
| sr7609 | 3 |
| sr7610 | 2 |
| sr7611 | 2 |
| sr7612 | 2 |
| sr7614 | 3 |
| sr7615 | 3 |
| sr7616 | 3 |
| sr7618 | 2 |

### Example 3: Evaluation of Compounds of the Invention on the Wild-Type Phenotype of Reference Strains Gram+ et Gram−

A screening of molecules of the invention on several wild-type phenotype strains has been carried out.

The strains initially selected are:
Gram+: Streptococcus equi subsp zooepidemicus and Staphylococcus aureus
Gram−: Escherichia coli, Klebsiella pneumoniae and Stenotrophomonas maltophila.

The results are outlined in the table below:

| Molecules | S. zooepidemicus | S. aureus | E. coli | K. pneumoniae | S. maltophila |
|---|---|---|---|---|---|
| sr7583 | | | 2 | 2 | |
| sr7585 | 3 | 3 | 3 | 2 | 2 |
| sr7609 | | 2 | 2 | 2 | 2 |
| sr7614 | 2 | 2 | 3 | 2 | 2 |
| sr7615 | 3 | 3 | 3 | 2 | 2 |
| sr7616 | 3 | 2 | 3 | 3 | 2 |

### Example 4: Evaluation of Compounds of the Invention on Strains Presenting High Levels of Resistance

Compounds of the invention were tested on 4 strains of E. coli, 3 strains of K. pneumoniae and 4 strains of S. aureus with the resistance profiles below.

| ATB Gram− | | AMX | AMC | CEF | CEQ | COL | STP | K | GM | AN | TET | RA | SUL | FLU | ENO | MAR |
|---|---|---|---|---|---|---|---|---|---|---|---|---|---|---|---|---|
| Escherichia | Strain 1 | R | R | R | R | S | R | R | R | S | R | R | R | R | R | R |
| coli | Strain 2 | R | R | R | R | S | R | R | R | S | R | R | R | R | R | R |
| | Strain 3 | R | R | R | R | S | R | R | R | S | R | R | R | R | R | R |
| | Strain 4 | R | R | R | R | S | R | R | R | S | R | R | R | R | R | R |

| ATB Gram− | | AMC | CEF | CEQ | COL | STP | K | GM | AN | TET | SUL | FLU | ENO | MAR |
|---|---|---|---|---|---|---|---|---|---|---|---|---|---|---|
| Klebsiella | Strain 1 | S | R | R | S | R | R | R | S | R | R | S | S | S |
| pneumoniae | Strain 2 | S | S | S | S | R | S | S | S | R | R | R | R | S |
| | Strain 3 | R | R | R | S | R | S | S | S | R | S | S | S | S |

| ATB Gram+ | | PEN | AMX | AMC | CEF | CEQ | STP | K | GM | TET | E | RA | SUL | ENO | MAR |
|---|---|---|---|---|---|---|---|---|---|---|---|---|---|---|---|
| Staphylococcus | Strain 1 | R | R | R | R | R | S | R | R | R | R | S | S | R | R |
| aureus | Strain 2 | R | R | R | R | R | S | R | R | R | R | S | S | R | R |
| | Strain 3 | R | R | R | R | R | S | R | R | R | S | S | S | R | R |
| | Strain 4 | R | R | R | R | R | S | R | R | R | R | S | S | R | R |

PEN: penicillin; AMX: amoxicillin; AMC: amoxicillin-clavulanic acid; CEF: ceftiofur; CEQ: cefquinome; COL: Colistin, STP: streptomycin; K: kanamycin; GM: gentamycin; AN: Amikacin; TET: tetracycline; E: erythromycin; RA: rifampicin; SUL: sulfamide; ENO: enrofloxacin; MAR: marbofloxacin; FLU: flumequine.

The molecules were tested at concentrations (mg/ml) of 1; 0.5 and 0.1 compared with the triclosan used at 0.1 mg/ml.

The results obtained on the strains of E. coli are the following:

| | | Concentration | | Effects |
|---|---|---|---|---|
| E. coli | Molecules | (mg/ml) | Δ DO | (0 < 1 < 2 < 3) |
| Strain 1 | Triclosan | 0.1 | 1.005 | 3 |
| | sr7585 | 1 | 0.920 | 3 |
| | | 0.5 | 0.746 | |
| | | 0.1 | 0.196 | |
| | sr7615 | 1 | 0.896 | 2 |
| | | 0.5 | 0.777 | |
| | | 0.1 | 0.047 | |
| | sr7616 | 1 | 0.857 | 2 |
| | | 0.5 | 0.640 | |
| | | 0.1 | 0.089 | |
| Strain 2 | Triclosan | 0.1 | 1.197 | 3 |
| | sr7585 | 1 | 1.097 | 3 |
| | | 0.5 | 0.964 | |
| | | 0.1 | 0.313 | |
| | sr7615 | 1 | 1.082 | 2 |
| | | 0.5 | 0.886 | |
| | | 0.1 | 0.080 | |
| | sr7616 | 1 | 1.069 | 3 |
| | | 0.5 | 0.854 | |
| | | 0.1 | 0.331 | |
| Strain 3 | Triclosan | 0.1 | 0.948 | 3 |
| | sr7585 | 1 | 0.893 | 3 |
| | | 0.5 | 0.736 | |
| | | 0.1 | 0.186 | |
| | sr7615 | 1 | 0.882 | 2 |
| | | 0.5 | 0.526 | |
| | | 0.1 | 0.029 | |
| | sr7616 | 1 | 0.825 | 3 |
| | | 0.5 | 0.679 | |
| | | 0.1 | 0.141 | |
| Strain 4 | Triclosan | 0.1 | 1.080 | 3 |
| | sr7585 | 1 | 0.979 | 3 |
| | | 0.5 | 0.848 | |
| | | 0.1 | 0.322 | |
| | sr7615 | 1 | 0.945 | 3 |
| | | 0.5 | 0.809 | |
| | | 0.1 | 0.253 | |
| | sr7616 | 1 | 0.853 | 3 |
| | | 0.5 | 0.868 | |
| | | 0.1 | 0.308 | |

The results obtained on the strains of K. pneumoniae are the following:

| | | Concentration | | Effects |
|---|---|---|---|---|
| K. pneumoniae | Molecules | (mg/ml) | Δ DO | (0 < 1 < 2 < 3) |
| Strain 1 | Triclosan | 0.1 | 0.824 | 3 |
| | sr7585 | 1 | 0.644 | 3 |
| | | 0.5 | 0.595 | |
| | | 0.1 | 0.251 | |
| | sr7615 | 1 | 0.640 | 2 |
| | | 0.5 | 0.490 | |
| | | 0.1 | 0.025 | |
| | sr7616 | 1 | 0.646 | 3 |
| | | 0.5 | 0.594 | |
| | | 0.1 | 0.236 | |
| Strain 2 | Triclosan | 0.1 | 0.893 | 3 |
| | sr7585 | 1 | 0.701 | 2 |
| | | 0.5 | 0.620 | |
| | | 0.1 | 0.035 | |
| | sr7615 | 1 | 0.621 | 2 |
| | | 0.5 | 0.518 | |
| | | 0.1 | −0.021 | |
| | sr7616 | 1 | 0.686 | 2 |
| | | 0.5 | 0.641 | |
| | | 0.1 | 0.151 | |
| Strain 3 | Triclosan | 0.1 | 1.092 | 3 |
| | sr7585 | 1 | 0.872 | 2 |
| | | 0.5 | 0.683 | |
| | | 0.1 | 0.265 | |
| | sr7615 | 1 | 0.913 | 2 |
| | | 0.5 | 0.598 | |
| | | 0.1 | 0.134 | |
| | sr7616 | 1 | 0.847 | 2 |
| | | 0.5 | 0.652 | |
| | | 0.1 | 0.213 | |

The results obtained on the strains of S. aureus are the following:

| | | Concentration | | Effects |
|---|---|---|---|---|
| S. aureus | Molecules | (mg/ml) | Δ DO | (0 < 1 < 2 < 3) |
| Strain 1 | Triclosan | 0.1 | 0.441 | 3 |
| | sr7585 | 1 | 0.445 | 3 |
| | | 0.5 | 0.445 | |
| | | 0.1 | 0.352 | |
| | sr7615 | 1 | 0.428 | 3 |
| | | 0.5 | 0.434 | |
| | | 0.1 | 0.250 | |
| | sr7616 | 1 | 0.445 | 3 |
| | | 0.5 | 0.443 | |
| | | 0.1 | 0.276 | |
| Strain 2 | Triclosan | 0.1 | 0.459 | 3 |
| | sr7585 | 1 | 0.457 | 3 |
| | | 0.5 | 0.458 | |
| | | 0.1 | 0.385 | |
| | sr7615 | 1 | 0.382 | 3 |
| | | 0.5 | 0.412 | |
| | | 0.1 | 0.308 | |
| | sr7616 | 1 | 0.459 | 3 |
| | | 0.5 | 0.458 | |
| | | 0.1 | 0.303 | |
| Strain 3 | Triclosan | 0.1 | 0.532 | 3 |
| | sr7585 | 1 | 0.530 | 3 |
| | | 0.5 | 0.529 | |
| | | 0.1 | 0.459 | |
| | sr7615 | 1 | 0.445 | |
| | | 0.5 | 0.482 | 3 |
| | | 0.1 | 0.310 | |
| | sr7616 | 1 | 0.525 | 3 |
| | | 0.5 | 0.529 | |
| | | 0.1 | 0.299 | |
| Strain 4 | Triclosan | 0.1 | 0.490 | 3 |
| | sr7585 | 1 | 0.486 | 3 |
| | | 0.5 | 0.491 | |
| | | 0.1 | 0.405 | |
| | sr7615 | 1 | 0.418 | 3 |
| | | 0.5 | 0.450 | |
| | | 0.1 | 0.351 | |
| | sr7616 | 1 | 0.491 | 3 |
| | | 0.5 | 0.491 | |
| | | 0.1 | 0.291 | |

In conclusion, compounds of the invention have revealed to have MICs (Minimal Inhibitory Concentrations) of the same magnitude as triclosan on multi-resistant strains.

### Example 4: In Vitro Cytotoxicity Tests

### Cell Culture and Cell Proliferation Tests

The human cell strain KB (epidermoid carcinoma) was obtained from the NCI (National Cancer Institute) and cultivated in the D-MEM medium added with 10% of foetal bovine serum (Invitrogen) in the presence of penicillin, streptomycin and fungizone in flasks of 75 cm³ under CO₂ at 5%. The cells were deposited on tissue culture microplates with 96 wells and a density of 650 cells/well in 200 μl of medium and treated 24 hours later with compounds of the invention dissolved in DMSO at concentrations going from 0.5 nM to 10 μM using a Biomek 3000 (Beckman-Coulter) automaton. The controls received the same volume of DMSO (1% of final volume). After 72 hours of exposure, the MTS reagent (Celltiter 96Aqueous One solution, Promega) was added and incubated for 3 hours at 37° C.: the absorbance was measured at 490 nm and the results of inhibition of the cell proliferation were calculated according to the ratio [(1−(OD490treated/OD490control))×100].

Results: Cytotoxicity at 10⁻⁵ and 10⁻⁶M on Triplicate KB Cells, Measured after 72 Hours of Exposure.

The percentage of inhibition of cell growth with respect to the cells receiving the same quantity of DMSO is outlined in the table below.

| | 10⁻⁵ M | 10⁻⁶ M |
|---|---|---|
| Triclosan | 78 ± 3 | 5 ± 12 |
| sr7580 | 16 ± 4 | 5 ± 8 |
| sr7581 | 0 ± 6 | 0 ± 6 |
| sr7582 | 1 ± 7 | 0 ± 9 |
| sr7583 | 0 ± 7 | 0 ± 8 |
| sr7584 | 0 ± 6 | 0 ± 9 |
| sr7585 | 0 ± 5 | 0 ± 6 |
| sr7586 | 0 ± 2 | 6 ± 8 |
| sr7587 | 0 ± 4 | 8 ± 4 |
| sr7607 | 0 ± 2 | 0 ± 5 |
| sr7608 | 7 ± 11 | 0 ± 14 |
| sr7614 | 6 ± 4 | 0 ± 3 |
| sr7615 | 0 ± 3 | 0 ± 4 |
| sr7616 | 0 ± 9 | 0 ± 8 |

This study has made it possible to highlight an absence of marked cytotoxicity of molecules of the invention, in the concentration range corresponding to their use.

### Example 5: Affinity Tests for Androgenic Receptors

The affinity of Triclosan (reference compound) and of the compound sr7615 of the present invention for androgenic receptors was measured by the percentage of inhibition of the bonding of a radioactive marked ligand (testosterone) specific to the androgenic receptors.

### Results

In the context of this example, results showing an inhibition greater than 50% are considered as representing significant effects of a compound.

The results obtained are presented in the table below.

| | Concentration | % of inhibition (average of |
|---|---|---|
| Compound | tested | the two measurements) |
| Triclosan | 10⁻⁵ M | 79.8 |
| Sr7615 | 10⁻⁵ M | 3.1 |

Also, Triclosan bonds itself significantly to the androgenic receptors (endocrine disruptor), contrary to the compound of the present invention.

1. A method for biocidal treatment of a substrate comprising a step of contacting said substrate with a compound of following formula (I): 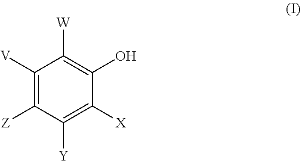
wherein:
V represents:
a group —OR, wherein R is a linear or branched alkyl in C₁ to C₄, optionally substituted by at least one halogen, R being in particular a methyl or a perhalogenoalkyl in C₁ to C₄; or
I;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
at least one of W, X, Y and Z representing a halogen,
the following compound being excluded: 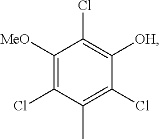
as well as its pharmaceutically acceptable salts.

2. The method according to claim 1, wherein V represents I, and in particular:
W and/or Y represent H; and/or
X and/or Z represent a halogen, in particular F.

3. The method according to claim 1 of a compound of following formula (Ia): 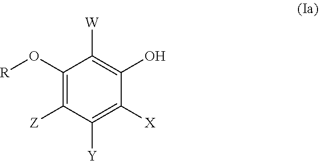
wherein:
R is a linear or branched alkyl in C₁ to C₄, optionally substituted by at least one halogen, R being in particular a methyl or a perhalogenoalkyl in C₁ to C₄;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
at least one of W, X, Y and Z representing a halogen,
the following compound being excluded:

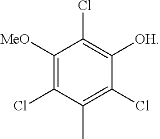

4. The method according to claim 1, wherein:
two at least of W, X, Y and Z representing a halogen, in particular two at least of X, Y and Z representing a halogen;
X is chosen from the halogens, and at least one of Y and of Z is chosen from the halogens;
X and Z are each independently chosen from the halogens, Y representing in particular H or a halogen; or
X and Y are each independently chosen from the halogens Z representing in particular H or a halogen;
X, Y and Z are each independently chosen from the halogens.

5. The method according to claim 1, wherein W is chosen from H and the linear or branched alkyls in C₁ to C₄, optionally substituted by at least one halogen, R being in particular H, a methyl or a perhalogenoalkyl in C₁ to C₄.

6. The method according to claim 1, wherein:
W represents H; and
X, Y and Z are each independently chosen from the halogens; where
X and Z represent Br and Cl, X representing Br and Z representing Cl, or X representing Cl and Z representing Br, Y representing in particular H; or
X and Y represents a halogen, Z representing in particular H;
provided that:
if Z represents F, then at least one of X and of Y is chosen from F, Br and I; and Y is chosen from Cl, Br and I.

7. The method according to claim 1, wherein said compound is chosen from the group formed by compounds of following structure:

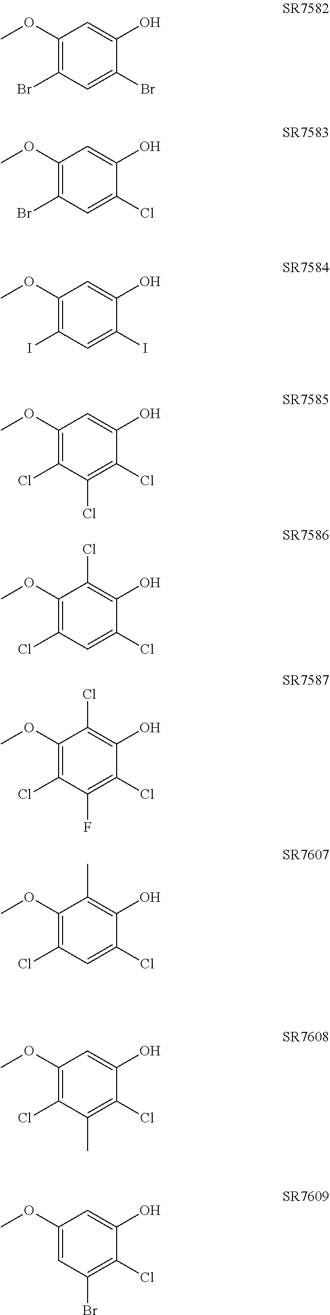

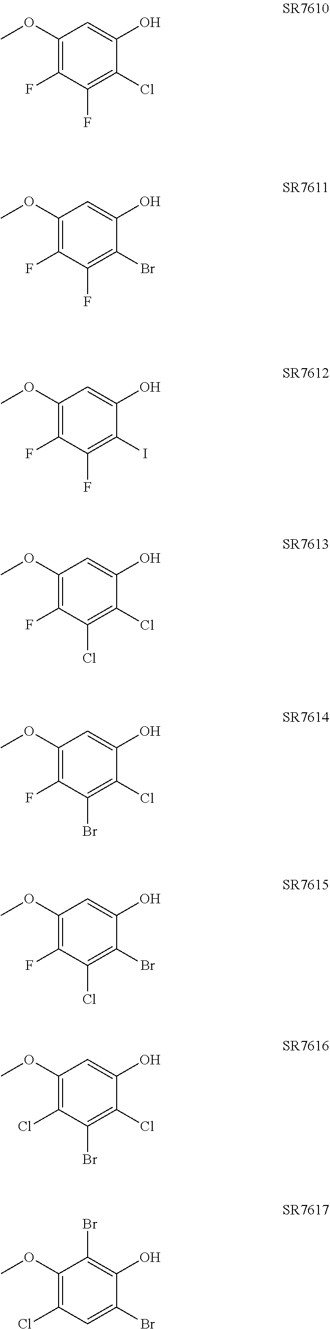

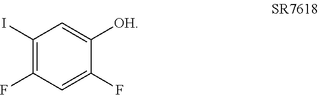

8. The method according to claim 1 wherein the biocidal treatment is an antibacterial treatment.

9. The method according to claim 1 wherein the biocidal treatment is a disinfection of the substrate, said substrate being a fluid or a surface.

10. A method of antimicrobial treatment of a human or an animal comprising topical administration to the human or animal in need thereof of an effective amount of a compound of formula (I) or (Ia) such as defined in claim 1.

11. A compound of following formula (II): 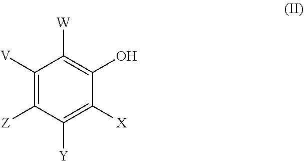
wherein:
V represents:
a group —OR wherein R is a linear or branched alkyl in C₁ to C₄, optionally substituted by at least one halogen, R being in particular a methyl or a perhalogenoalkyl in C₁ to C₄; or
I;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
provided that at least two of W, X, Y and Z represent a halogen;
as well as its pharmaceutically acceptable salts;
said compound not being one of the following formulae:

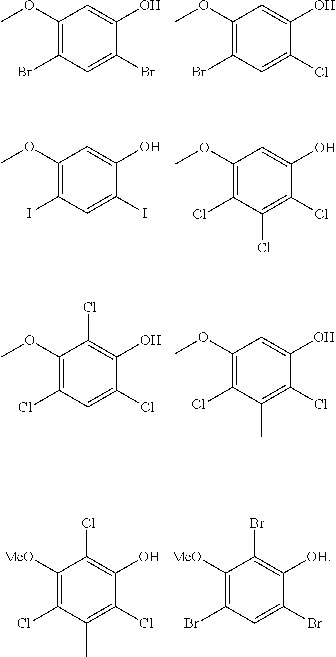

12. The compound according to claim 11, of following formula (IIa): 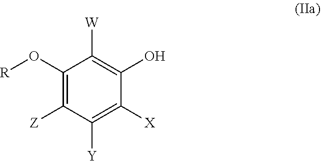
wherein:
R is a linear or branched alkyl in C₁ to C₄, optionally substituted by at least one halogen, R being in particular a methyl or a perhalogenoalkyl in C₁ to C₄;
W, X, Y and Z are each independently chosen from H, the halogens and the linear or branched alkyls in C₁ to C₄, optionally substituted by at least one halogen, W, X, Y and Z being in particular each independently H, a methyl, F, Cl, Br, I or a perhalogenoalkyl in C₁ to C₄;
provided that:
X, Y and Z are each independently chosen from the halogens; or
at least one of W and of Y, in particular W, does not represent H;
said compound not being one of the following formulae:

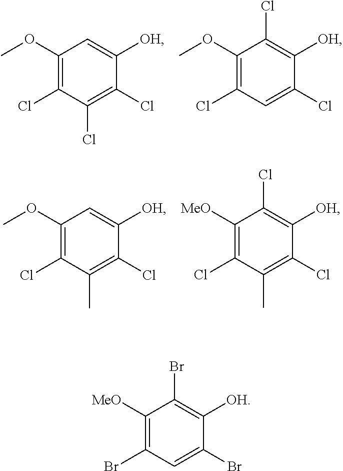

13. A pharmaceutical composition comprising as active substance, a compound of formula (II) or (IIa) such as defined in claim 11, and a pharmaceutically acceptable excipient.
